(19) United States
(12) Patent Application Publication
(10) Pub. No.: US 2010/0055175 A1
(43) Pub. Date: Mar. 4, 2010

(54) SOFT GELATIN CAPSULE SHELLS CONTAINING OIL SOLUBLE FLAVORING AND METHODS OF MAKING THE SAME

(76) Inventor: James Nugent, Saskatoon (CA)

Correspondence Address: LAHIVE & COCKFIELD, LLP;FLOOR 30, SUITE 3000, ONE POST OFFICE SQUARE, BOSTON, MA, 02109, US

(21) Appl. No.: 12/526,132

(22) PCT Filed: Mar. 6, 2008
(86) PCT No.: PCT/CA08/00427

§ 371 Date: Aug. 6, 2009

Related U.S. Application Data
(60) Provisional application No. 60/905,212, filed on Mar. 6, 2007.

(51) Int. Cl. A61K 9/48; A61K 47/42; A61P 43/00

(52) U.S. Cl. 424/456; 514/774

(57) ABSTRACT

A novel gelatin capsule shell having reduced hardness and enhanced chewability has now been discovered. The present invention is based, at least in part, on the finding that soft gelatin capsule shells can be produced, for example, by the incorporation of oil soluble flavoring and increased levels of plasticizer such as glycerin.

### RELATED APPLICATIONS

The present invention claims priority to U.S. Application No. 60/905212, the contents of which are hereby incorporated by reference herein.

### FIELD OF INVENTION

The present invention relates generally to soft gelatin capsule formulations, medicinal and nutritive dose encapsulations and methods of making the same.

### BACKGROUND

Gelatin capsules are comprised of a gelatin sheath encapsulating a fill of a medicament, vitamin or other material to be consumed by the user. While a variety of such capsules have been described in the prior art, see, for example, U.S. Pat. Nos. 6,258,380 and 6,346,231, skilled artisans have been unable to design capsules with reduced hardness and enhanced chewability.

### SUMMARY OF THE INVENTION

The present invention provides gelatin capsule shells and gelatin capsules with enhanced flavor profiles and enhanced texture and aesthetic features, so as to increase patient compliance, appeal and acceptance. In particular, the present invention is directed to gelatin capsule shells and gelatin capsules with a reduced hardness and enhanced chewability. The present invention further provides methods of manufacturing the same.

In one aspect, the present invention is directed to a gelatin capsule shell including an oil soluble flavoring agent for use in a pharmaceutical or nutraceutical composition. In various embodiments, the shell has a hardness of less than 4, 3, 2 or 1 Newtons (as measured on a Bareiss hardness tester) or of between 0.5 and 4 Newtons, between 0.5 and 3 Newtons or between 0.75 and 2 Newtons. In a particular embodiment, the shell includes gelatin and a plasticizer, for example, glycerin, propylene glycol, polyethylene glycol, sorbitol, maltitol and combinations thereof In a particular embodiment, the plasticizer is glycerin. In certain embodiments, the gelatin and plasticizer are present at a ratio of between 1.5:1 to 0.5:1, of between 1.25:1 to 0.75:1 or of between 1.1:1 to 0.9:1. In certain embodiments, the shell includes between 25% and 60% by weight, between 35% and 55% by weight, or between 40% and 50% by weight of plasticizer.

In various embodiments, the shell may be semi-transparent or semi-opaque. In particular embodiments, the shell includes between about 0.01% and about 15% by weight, between about 0.1% and about 12% by weight, between about 0.4% and about 10% by weight or between about 1% and about 8% by weight of oil soluble flavoring agent. In a particular embodiment, the shell includes between 2% and 6% by weight of oil soluble flavoring agent. Optionally, the oil soluble flavoring agent may have a flavor selected from the group consisting of grape, apple, cinnamon, berry, strawberry, orange, cherry, lemon, lime, raspberry, peach, grapefruit, mango, guava, mint, chocolate, coca, vanilla, lemon, nut, almond, coconut, blueberry, blackberry, banana, pineapple and combinations thereof. In various embodiments, the oil soluble flavoring agent may be either a natural or artificial flavoring agent, or a combination thereof.

In one embodiment, the shell includes between about 25% and about 45% by weight, between about 35% and about 45% by weight, or between about 40% and about 45% by weight of gelatin. Alternatively, or in addition, the gelatin may have a single bloom of between about 100 and about 275, between about 150 and about 200, or between about 160 and 180.

In other embodiments, the shell includes at least one of a plasticizer, water, a disintegrant, a sweetening agent and colorant, for example, a water soluble colorant.

In certain embodiments, the shell includes a disintegrant selected from the group consisting of cellulose, cellulose derivative, starch, starch derivative, gum, gum derivative, clay, polysaccharide, polyvinylpyrrolidone derivative and silica and combinations thereof. In various embodiments, the shell comprises between about 1% and about 10% by weight, between about 2.5% and about 7.5% by weight, or between about 4% and 6% by weight of disintegrant.

In another embodiment, the shell includes between about 1% and about 15% by weight, between about 3% and about 10% by weight or between about 5% and 7% by weight of water (as calculated from loss-on-drying).

In yet another embodiment, the shell includes a sweetening agent selected from the group consisting of monosaccharide sugars, disaccharide sugars, polysaccharide sugars, sugar alcohols, mannitol, xylitol, maltitol, sorbitol, synthetic sugar substitutes, aspartame, saccharine, sodium sacchrinate, acesulpham K, neohesperidin hydrochloride, sodium cyclamate, naturally occurring sugar substitutes, sucralose, and combinations thereof. For example, the shell may include between about 0.1% and about 25% by weight, between about 5% and about 20% by weight, or between about 10% wt and about 15% weight of sweetening agent (as calculated from the dry weight basis).

In one embodiment the shell is a chewable shell. In yet another embodiment, the shell is colored, for example, the shell may include a natural colorant, a synthetic colorant, or a combination thereof.

In another aspect, the invention is directed to a gelatin capsule comprising a shell as described herein.

In another aspect, the invention is directed to a method of making a shell or gelatin capsule as described herein. In another aspect, the invention is directed to a method of making a gelatin capsule shell mass by heating a mixture of gelatin and a plasticizer, for example, glycerin, propylene glycol, polyethylene glycol, sorbitol, maltitol and combinations thereof, under vacuum; and adding an oil soluble flavoring agent to the heated mixture. The mixture can include gelatin and plasticizer at a ratio of between 1.5:1 to 0.5:1, between 1.25:1 to 0.75:1 or between 1.1:1 to 0.9:1.

In other embodiments, the gelatin capsule shell mass comprises between 0.01% and 15%, between 0.1% and 12%, between 0.4% and 10%, between 1% and 8% or between 2% and 6% by weight of oil soluble flavoring agent. In yet another embodiment, the method includes adding a disintegrant to the gelatin and glycerin mixture, wherein the disintegrant is selected from the group consisting of cellulose, cellulose derivative, starch, starch derivative, gum, gum derivative, clay, polysaccharide, polyvinylpyrrolidone derivative, silica and combinations thereof. For example, the gelatin capsule shell mass may include between 1% and 10% by weight, between 2.5% and 7.5% by weight, or between 4% and 6% by weight of disintegrant. In a particular embodiment, the disintegrant is cellulose.

### DETAILED DESCRIPTION OF INVENTION

A novel gelatin capsule shell having reduced hardness and enhanced chewability has now been discovered. The present invention is based, at least in part, on the finding that soft gelatin capsule shells, for example, of less than about 4 Newtons, can be produced, for example, by the incorporation of oil soluble flavoring and increased levels of plasticizer such as glycerin. The present invention is based, at least in part, on the unexpected finding that the incorporation of an oil soluble flavoring agent can serve as a lubricant to prevent clumping and stickiness between layers of gelatin capsule shells, which would otherwise be expected for soft gelatin capsules of reduced hardness and enhanced chewability. In addition, the incorporation of an oil soluble flavoring agent has been found to unexpectedly enhance the “machining” and “manufacturing” capability of the gelatin capsule shell mass. For example, the incorporation of the oil soluble flavoring agent serves to strengthen the gelatin capsule shell mass, thereby allowing it to stretch without ripping. Such feature is particularly important, for example, to allow the encapsulation machine to pull the gelatin mass ribbon from the casting drums during the manufacturing process, thereby unexpectedly allowing for the production of soft gelatin capsules of reduced hardness and enhanced chewability.

Moreover, the compositions of the present invention serve to enhance patient compliance by providing a soft and/or chewable capsule. Accordingly, such compositions are found to be more appealing to patients in general and, in particular, to younger and older patients who may find medication, for example, harder capsules, pills or tablets, inconvenient or may even be unable to swallow medication, for example, harder capsules, pills or tablets. By contrast, the compositions of the present invention are easily chewed or swallowed by patients, thereby improving patient compliance and acceptance.

Such features of the compositions of the present invention provide further advantages over the compositions of the prior art. For example, because the capsules are easily chewed or swallowed, the active ingredients can have more rapid effect. Indeed, upon rupturing of the capsule, or alternatively, the dissolving of the gelatin capsule by acids of the digestive and/or gastrointestinal tract, the active ingredients of the compositions are more readily accessible to the body.

In addition, the present invention is directed to a novel gelatin capsule including an oil soluble flavoring agent. The present invention involves the incorporation of an oil soluble flavoring agent in the gel capsule shell so as to, in part, allow for reduced hardness and enhanced chewability, as described above and, further, to improve patient compliance and acceptance by improving the flavor profile of the capsule, for example, by masking the unpleasant flavor and/or smell of certain fillings, active agents or ingredients generally, for example, the gelatin forming the shell itself or part of the fill.

In one aspect, the present invention is directed to a gelatin capsule shell having reduced hardness and enhanced chewability. In various embodiments, the shell has a hardness of less than 4, 3.75, 3.5, 3.25, 3, 2.75, 2.5, 2.25, 2, 1.75, 1.5, 1.25, 1, 0.75 or 0.50 Newtons (as measured on a Bareiss hardness tester). Additionally, the shell may have a hardness of between 0.5 and 4 Newtons, between 0.5 and 3 Newtons, between 0.75 and 2 Newtons or between 0.75 and 1.50 Newtons. Ranges intermediate to the above recited amounts, e.g., about 3.8 Newtons, are also intended to be part of this invention. For example, ranges of values using a combination of any of the above recited values as upper and/or lower limits are intended to be included.

In one aspect the present invention provides a gelatin capsule shell including an oil soluble flavoring agent. As used herein, the term “oil soluble flavoring agent” refers to any agent that provides an appealing flavor and whose solubility in water is from very slightly soluble to insoluble. In a particular embodiment, the agent is insoluble in water. In general, when an oil soluble flavoring agent of the invention is mixed with water, two phases are formed, an oil phase and a water phase, with substantially all of the oil soluble flavoring agent being in the oil phase.

Oil soluble flavoring agents suitable for use in the present invention are well known in the art and include, but are not limited to, synthetic flavor oils, flavoring aromatics, oils, oleo resins and extracts derived from plants, leaves, flowers, fruits, and combinations thereof. These oil soluble flavoring agents are generally liquids but can also be used as spray dried solids, powdered solids and the like. Suitable oil soluble flavoring agents include, but are not limited to, spearmint oil, cinnamon oil, oil of wintergreen (methylsalicylate), peppermint oils, clove oil, bay oil, anise oil, eucalyptus oil, thyme oil, cedar leaf oil, oil of nutmeg, oil of sage, oil of bitter almonds, peanut butter flavor, chocolate flavor, rum flavor, cassia oil, cinnamon mint flavor, corn mint oil, cardamom flavor, ginger flavor, cola flavor, cherry cola flavor, and the like. Oil soluble flavoring agents such as aldehydes and esters including, but not limited to, cinnamyl acetate, cinnamaldehyde, citral diethylacetal, dihydrocarvyl acetate, eugenyl formate and p-methylanisole may also be used. Generally any oil soluble flavoring agent such as those described in Chemicals Used in Food Processing, pub 1274 by the National Academy of Sciences, pages 63-258 may be used.

The oil soluble flavoring agents may provide the composition with any appealing flavor, for example, that serves to mask an undesirable or unpleasant flavor or odor of the composition. In certain embodiments, the oil soluble flavoring agents may provide the composition with grape, apple, cinnamon, berry, strawberry, orange, cherry, lemon, lime, raspberry, peach, grapefruit, mango, guava, mint, chocolate, coca, vanilla, lemon, nut, almond, coconut, blueberry, blackberry, banana, pineapple, spearmint, wintergreen, peppermint, clove, bay, anise, eucalyptus, thyme, nutmeg, sage, bitter almonds, cinnamon mint, corn mint, cardamom, ginger, cola, rum, peanut butter and/or chocolate flavors. In other embodiments, blends of these flavors may be utilized.

The oil soluble flavoring agents should be present in an amount sufficient to provide a distinct flavor perception on the part of the patient. In a particular embodiment, the flavoring agent should be present in an amount sufficient to mask an otherwise undesirable or unpleasant flavor of the capsule composition, for example, that of the active agent or of the gelatin forming the shell or incorporated into the fill. In another embodiment, the oil soluble flavoring agent is present in an amount sufficient to provide a sustained flavor perception on the part of the patient. For example, the flavor should be present in an amount sufficient to mask the undesirable or unpleasant flavor of the composition during the period that the composition is retained in the mouth.

In one embodiment, the shell includes at least about 0.01%, 0.05%, 0.10%, 0.20%, 0.30%, 0.40%, 0.50%, 0.75%, 1%, 1.25%, 1.5%, 1.75%, 2%, 2.25%, 2.5%, 2.75%, 3%, 3.25%, 3.50%, 3.75%, 4%, 4.25%, 4.5%, 4.75%, 5%, 5.25%, 5.5%, 5.75%, 6%, 6.25%, 6.50%, 6.75%, 7%, 7.25%, 7.5%, 7.75%, 8%, 8.25%, 8.5%, 8.75%, 9%, 9.25%, 9.50%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 16%, 17%, 18%, 19%, 20%, 21%, 22%, 23%, 24% and 25% by weight of oil soluble flavoring agent (as calculated from the dry weight basis). In particular embodiments, the shell includes between about 0.01% and about 15% by weight, between about 0.4% and about 10%, between about 1% and about 8%, or between about 2% and 6% by weight of oil soluble flavoring agent. Ranges intermediate to the above recited amounts, e.g., about 3.3% to about 9.6% by weight of oil soluble flavoring agent, are also intended to be part of this invention. For example, ranges of values using a combination of any of the above recited values as upper and/or lower limits are intended to be included.

The capsule shells of the compositions of the present invention are formed, at least in part, of gelatin. One skilled in the art will appreciate that any of a variety of gelatins may be used in the compositions of the present invention including, but not limited to, porcine gelatin, bovine gelatin and fish gelatin. The gelatin in the gelatin shell should be present in an amount sufficient to provide sufficient structure to the capsule. In particular embodiments, the gelatin is present in an amount suitable to render the capsule chewable.

In one embodiment, the shell includes at least about 10%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% and 50% by weight of gelatin (as calculated from the dry weight basis). In particular embodiments, the shell includes between about 25% and about 45% by weight, between about 35% and about 45% or between about 40% and about 45% by weight of gelatin. Ranges intermediate to the above recited amounts, e.g., about 13% to about 43% by weight of gelatin, are also intended to be part of this invention. For example, ranges of values using a combination of any of the above recited values as upper and/or lower limits are intended to be included.

In various embodiments, the gelatin has a bloom of at least about 75, 100, 110, 120, 130, 140, 150, 160, 170, 180, 190, 200, 210, 220, 230, 240, 250 and 275. In a particular embodiment, the gelatin has a single bloom of between about 100 and about 275, between about 150 and about 200, or between about 160 and 180. Ranges intermediate to the above recited amounts, e.g., about 112 to about 173, are also intended to be part of this invention. For example, ranges of values using a combination of any of the above recited values as upper and/or lower limits are intended to be included. As is well known in the art, bloom is a measure of the cohesive strength of gelatin. Bloom is determined with the use of a texture analyzer that probes into gelatin to define, in part, the average molecular weight of the gelatin.

In certain embodiments, the gelatin shell of the invention includes at least one of a plasticizer, water, a disintegrant, a sweetening agent and a colorant. In a particular embodiment, the gelatin shell includes each of, but is not limited to, plasticizer, water, disintegrant, sweetening agent and colorant, for example, a water soluble colorant.

In certain embodiments, the gelatin shells of the invention include a plasticizer. As used herein, the term “plasticizer” refers to a substance that modifies the flexibility and diffusion properties of the composition. Suitable plasticizers for use in the present invention include, but are not limited to, glycerin, propylene glycol, polyethylene glycol, sorbitol, maltitol and combinations thereof. In a particular embodiment, glycerin is utilized as a plasticizer.

In one embodiment, the shell includes at least about 10%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 70% and 75% by weight of plasticizer (as calculated from the dry weight basis). In particular embodiments, the shell includes between about 25% and about 60% by weight, between about 35% and about 55% by weight or between about 40% and about 50% by weight of plasticizer. Ranges intermediate to the above recited amounts, e.g., about 13% to about 68% by weight of plasticizer, are also intended to be part of this invention. For example, ranges of values using a combination of any of the above recited values as upper and/or lower limits are intended to be included.

In certain embodiments, the gelatin and plasticizer, for example, glycerin, are present at a ratio of between 1.5:1 to 0.5:1, of between 1.25:1 to 0.75:1 or of between 1.10:1 to 0.9:1. Ranges intermediate to the above recited amounts, e.g., between 1.4:1 to 0.8:1 are also intended to be part of this invention. For example, ranges of values using a combination of any of the above recited values as upper and/or lower limits are intended to be included.

In another embodiment of the invention, the gelatin shells include a disintegrant. As used herein, the term “disintegrant” refers to a substance that serves to promote the breaking apart of the composition upon ingestion. Suitable disintegrants for use in the present invention include, but are not limited to, cellulose, cellulose derivatives, starch, starch derivatives, gum, gum derivatives, clay, polysaccharide, polyvinylpyrrolidone derivative, silica and combinations thereof.

In one embodiment, the shell includes at least about 0.10%, 0.20%, 0.30%, 0.40%, 0.50%, 0.75%, 1%, 1.25%, 1.5%, 1.75%, 2%, 2.25%, 2.5%, 2.75%, 3%, 3.25%, 3.50%, 3.75%, 4%, 4.25%, 4.5%, 4.75%, 5%, 5.25%, 5.5%, 5.75%, 6%, 6.25%, 6.50%, 6.75%, 7%, 7.25%, 7.5%, 7.75%, 8%, 8.25%, 8.5%, 8.75%, 9%, 9.25%, 9.50%, 9.75%, 10%, 10.25%, 10.5%, 10.75%, 11%, 11.25%, 11.5%, 11.75%, 12%, 12.25%, 12.5%, 12.75%, 13%. 13.25%, 13.5%, 13.75%, 14%, 14.25%, 14.5%, 14.75% and 15% by weight of disintegrant (as calculated from the dry weight basis). In particular embodiments, the shell includes between about 1% and about 10% by weight, between about 2.5% and about 7.5% by weight or between about 4% and about 5% by weight of disintegrant. Ranges intermediate to the above recited amounts, e.g., about 0.25% to about 14.8% by weight of disintegrant, are also intended to be part of this invention. For example, ranges of values using a combination of any of the above recited values as upper and/or lower limits are intended to be included.

The gelatin shells of the invention may further include a sweetening agent, for example, to enhance the taste of the final composition. Suitable sweetening agents for use in the present invention include, but are not limited to, xylitol, monosaccharide sugars, disaccharide sugars, polysaccharide sugars, sugar alcohols (for example, mannitol, maltitol or sorbitol), synthetic sugar substitutes (for example, aspartame, saccharine, sodium sacchrinate, acesulpham K, neohesperidin hydrochloride and sodium cyclamate), natural occurring sugar substitutes (for example, sucralose) and combinations thereof. In a particular embodiment, a single sweetening agent is utilized. Alternatively, in another embodiment, multiple sweetening agents may be used.

In one embodiment, the shell includes at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% by weight of sweetening agent (as calculated from the dry weight basis.). In particular embodiments, the shell includes between about 0.1% and about 25% by weight, between about 5% and about 20% by weight or between about 10% and about 15% by weight of sweetening agent. Ranges intermediate to the above recited amounts, e.g., about 2.5% to about 14.5% by weight of sweetening agent, are also intended to be part of this invention. For example, ranges of values using a combination of any of the above recited values as upper and/or lower limits are intended to be included.

In yet another embodiment of the invention, the gelatin shell includes water. The water serves, in part, as a processing aid. Additionally, the incorporation of water serves to provide the shell flexibility and elasticity. In a particular embodiment, water is added at the initial stages of the preparation of the gelatin shell. In such embodiments, the shell includes at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% by weight of water (as calculated from the molten mass basis). In particular embodiments, the shell includes between about 1% and about 40% by weight, between about 10% and about 35% by weight, between about 20% and about 30% by weight, or between about 25% and about 30% by weight of water. Ranges intermediate to the above recited amounts, e.g., about 12.5% to about 24.5% by weight of water, are also intended to be part of this invention. For example, ranges of values using a combination of any of the above recited values as upper and/or lower limits are intended to be included.

In particular embodiments, water may be removed after preparation of the shell by, for example, by drying. Accordingly, the shell may have a water content of at least about 1%, 2%, 3%, 4%, 5%, 6%, 7%, 8%, 9%, 10%, 11%, 12%, 13%, 14% and 15%, as calculated from a loss-on-drying basis. In a particular embodiment, the shell includes between about 1% and about 15% by weight, between about 3% and about 10% by weight or between about 5% and 7% by weight of water (as calculated from a loss-on-drying basis).

In certain embodiments, the gelatin shell may be uncolored. Alternatively, the shell can be colored, for example, to enhance the appeal of the final composition, thereby increasing patient acceptance. Any of a variety of colorants known in the industry may be utilized including, for example, natural or synthetic derivatives, or combinations thereof. In particular embodiments, the type and intensity of color is designed to enhance consumer acceptance. In another embodiment, the type and intensity of color is chosen so as to be in synergy with the flavoring agent.

In other embodiments, the gelatin shell may be semi-transparent. Alternatively, the gelatin shell may be semi-opaque.

The gelatin capsules of the present invention include a fill which can be present, for example, either as a clear liquid or a paste fill. In various embodiments, the capsule fill includes, but is not limited to, edible oil, thickening agent, sweetening agent, oil soluble flavoring agent and/or oil soluble colorant.

As used herein, the term “edible oil” is intended to include oils, either natural or synthetic. Particularly preferred are edible oils suitable for fish oil, flax oil, borage oil, evening primrose oil, wheat germ oil, hemp oil, echium oil and other vegetable, conjugated vegetable and animal oils. Omega 3, omega 6, omega 7, and/or omega 9 oils may be incorporated within the compositions of the present invention. The preferred edible oils are those that contain alpha linolenic acid (ALA), docasahexaenoic acid (DHA), eicosapentaenoic acid (EPA), stearidonic acid and gamma-linolenic acid (GLA), linolenic acid, erucic acid, almitoleic acid, oleic acid, palmitoelic acid, stearic acid, conjugated linolenic acids plus isomers and mixtures thereof.

In certain embodiments, the edible oil for use in the present invention is an omega 3 fatty acid, an omega 6 fatty acid, a monounsaturated fatty acid, a derivative thereof, and combinations thereof. As used herein, the term “omega 3 fatty acid” refers generally to a form of polyunsaturated fats including, but not limited to, docosahexaenoic acid (DHA), eicosapentacnoic acid (EPA), stearidonic acid, alpha linolenic acid (ALA), derivatives thereof, and combinations thereof. Other members of the omega 3 fatty acid family are well known in the art. In part, omega 3 fatty acids have been shown to lower plasma lipids and hyperglycemia, as well as to exhibit anti-inflammatory, vasodilator, antihypertensive and immunosuppressive effects.

As used herein, the term “omega 6 fatty acid” refers generally to another form of polyunsaturated fats including, but not limited to, linoleic acid (LA), gamma linolenic acid (GLA), arachidonic acid (AA), dihomogamma linolenic acid (DGLA), derivatives thereof, and combinations thereof. Other members of the omega 6 fatty acid family are well known in the art. These members, and their derivatives, are encompassed by the term, as used herein.

As used herein, the term “omega 7 fatty acid” includes, for example, palmitoelic acid.

As used herein, the term “omega 9 fatty acid” includes, for example, oleic and erucic acids.

As used herein, the term “monounsaturated fatty acid” generally includes, but is not limited to oleic acid, palmitolic acid, isomers (e.g., positional isomers) thereof, derivatives thereof, and combinations thereof. Numerous other members of the monounsaturated fatty acid family are well known in the art. These members, and their derivatives, are encompassed by the term “monosaturated fatty acid”.

The edible oil(s) are present in the composition at levels determined by various factors, including, but not limited to, the number of and nature of the components of the composition such as the fatty acids, the desired release profile of the various components, and the nutritional profile of the subject to whom the composition is administered. The edible oil should be generally present in the amount of at least 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg or 600 mg. In a particular embodiment, the edible oil is present at at least 250 mg. Ranges intermediate to the above recited amounts, e.g., about 113 to about 183 mg of edible oil, are also intended to be part of this invention. For example, ranges of values using a combination of any of the above recited values as upper and/or lower limits are intended to be included.

Alternatively, other active ingredients or medicaments may be incorporated in the gelatin capsules of the present invention. Indeed, the compositions of the present invention may include, for example, vitamins, minerals, herbs, botanicals, amino acids, enzymes, and metabolites.

In a particular embodiment, the present composition may optionally contain vitamins. Numerous vitamins exist, each playing a role in proper body functioning and health. The diet must contain vitamins as the human body is unable to synthesize them in adequate amounts. Non-limiting exemplary vitamins and their derivatives thereof for inclusion in the present compositions include vitamin A, B vitamins, vitamin C, vitamin D, vitamin E (e.g., DAT), vitamin K, vitamin H, folic acid and combinations thereof. These vitamins may be from any source or combination of sources, without limitation. Vitamins are present in an amount based on a variety of factors including, but not limited to, the number of and nature of the components of the composition such as the fatty acids, the desired release profile of the various components, and the nutritional profile of the subject to whom the composition is administered.

In another embodiment, the present composition may optionally contain minerals. Non-limiting exemplary minerals and their derivatives thereof for inclusion in the present compositions include iron, calcium, magnesium, potassium, copper, chromium (e.g., chromium chloride), zinc (e.g., zinc sulfate), choline (e.g., choline chloride) molybdenum, iodine, boron, selenium, manganese, and combinations thereof. These minerals may be from any source or combination of sources, without limitation. Minerals are present in an amount based on a variety of factors including, but not limited to, the number of and nature of the components of the composition such as the fatty acids, the desired release profile of the various components, and the nutritional profile of the subject to whom the composition is administered.

In particular embodiments, the capsule fill further includes at least one of a thickening agent, a sweetening agent and/or a flavoring agent. It has been found that the use of a thickening agent, sweetening agent and/or flavoring agent improved the organoleptic properties of the edible oil in the semi-transparent flavored, chewable capsule fill. The flavor, taste and aroma sensory responses, plus the physical attributes of mouth-feel can be varied by altering the ratio of ingredients within given ranges using the techniques described herein, or by methods familiar to a skilled artisan.

The thickening agent should be selected from the group consisting of edible waxes, either natural or synthetic, lecithins, wax esters, wax alcohols, fatty acids, fatty acid derivatives, white wax, paraffin wax, animal stearates, aluminum monostearate, ethylcellulose, polyethylene glycol 4000 & 6000, solid nonionics, solid glycol ester, acetylated monoglycerides, glycerin fatty acid esters, silicas, and mixtures thereof. The thickening agent may be present in the amount of at least 0.25%, 0.50%, 0.75%, 1%, 1.25%, 1.5%, 1.75%, 2%, 2.25%, 2.5%, 2.75%, 3%, 3.25%, 3.5%, 3.75% or 4% by weight. In a particular embodiment, the thickening agent is present at at least about 2.5%. Ranges intermediate to the above recited amounts, e.g., about 2.2% to about 3.6% by weight of thickening agent, are also intended to be part of this invention. For example, ranges of values using a combination of any of the above recited values as upper and/or lower limits are intended to be included.

Sweetening agents as described previously for use in the manufacture of the capsule shells may be used in the manufacture of the capsule fills. As described previously, suitable sweetening agents for use in the present invention include, but are not limited to, xylitol, monosaccharide sugars, disaccharide sugars, polysaccharide sugars, sugar alcohols (for example, mannitol, maltitol or sorbitol), synthetic sugar substitutes (for example, aspartame, saccharine, sodium sacchrinate, acesulpham K, neohesperidin hydrochloride and sodium cyclamate), natural occurring sugar substitutes (for example, sucralose) and combinations thereof. In a particular embodiment, a single sweetening agent is utilized. Alternatively, in another embodiment, multiple sweetening agents may be used.

In one embodiment, the fill includes at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% by weight of sweetening agent (as calculated from the dry weight basis). In particular embodiments, the fill includes between about 0.1% and about 25% by weight, between about 5% and about 20% by weight or between about 10% and about 15% by weight of sweetening agent. Ranges intermediate to the above recited amounts, e.g., about 2.5% to about 14.5% by weight of sweetening agent, are also intended to be part of this invention. For example, ranges of values using a combination of any of the above recited values as upper and/or lower limits are intended to be included.

In various embodiments, the capsule fill includes flavor agents, for example, water soluble or oil soluble flavoring agents. Oil soluble flavoring agents for use in the capsule fills of the present invention include those previously described as suitable for use in the manufacture of the capsule shells. For example, in particular embodiments, the flavoring agent is selected from the group consisting of oil soluble flavor and mixtures thereof, such as grape, apple, cinnamon, berry, strawberry, orange, cherry, lemon, lime, raspberry, peach, grapefruit, mango, guava, mint, chocolate, coca, vanilla, lemon, nut, almond, coconut, blueberry, blackberry, banana and pineapple. Moreover, in particular embodiments, the oil soluble flavoring agent is present at at least 0.4% by weight or at at least 4% by weight. The flavor agents, for example, oil soluble flavoring agents, should be chosen so as to impart pleasant experience in terms of flavor, aroma, mouth-feel and after-effects.

The fill may be uncolored. Alternatively, the fill may be colored, for example, to enhance the appeal of the final composition, thereby increasing patient acceptance. Any of a variety of colorants known in the industry may be utilized including, for example, natural or synthetic derivatives, or combinations thereof. In particular embodiments, the type and intensity of color is designed to enhance consumer acceptance. In another embodiment, the type and intensity of color is chosen so as to be in synergy with the flavoring agent. in another aspect, the invention is directed to a method of making a shell or gelatin capsule as described herein. For example, the present invention includes methods of making a gelatin capsule shell mass. As used herein, the term “gelatin capsule shell mass” refers to the blended gel mass utilized to form the shell, for example, upon encapsulation. The method may include the heating of a mixture of gelatin and a plasticizer, for example, glycerin, propylene glycol, polyethylene glycol, sorbitol, maltitol and combinations thereof, under vacuum; and adding an oil soluble flavoring agent to the heated mixture. In a particular embodiment, the oil soluble flavoring agent is added after vacuuming so as to prevent the vacuuming off of the agent.

Other ingredients, for example, disintegrant, water, sweetening agent and water soluble colorant may be added to the mixture as necessary, as described herein, and at the levels described herein.

### Exemplification

### Example 1

### Exemplary Method for Manufacture of Gelatin Capsule

Generally, capsules of the present invention may be manufactured by a variety of means known in the art. In a particular embodiment, capsules are manufactured in a rotary die process including certain or all of the following steps: (a) preparation of the soft gelatin mass; (b) preparation of soft gelatin fill; (c) encapsulation of the fill with the gelatin shell; (d) drying of the soft gelatin capsule; (e) inspection, sizing and polishing of soft gelatin capsule and (f) packaging of soft gelatin capsule.

### Step 1

As part of the first step for the manufacture of the flavored, chewable soft gelatin capsule, gelatin, plasticizer, sweetening agent and disintegrant were mixed with water. The mass was then heated and stirred under vacuum. The molten mass was then dropped into mobile vessels where oil soluble flavors and colorant were added using a turbine mixer that ensures consistency of the gelatin mass. This mass was kept at a constant temperature until it was needed for the encapsulation process.

As is known in the art, there are many types and styles of equipment to accomplish the preparation of the soft gelatin mass. The general selection of the equipment to be used in this step of the process can come from a wide variety of sources. Other commercial equipment having similar capabilities are known to those skilled in the art. It should be appreciated that such equipment may be suitable to provide all or part of the preparation of the present invention. The choice of equipment is dependent upon the batch size, heating, vacuum and economics of buying and operation. Preferred equipment for this invention is the gelatin reactor specifically designed for soft gelatin capsules.

### Step 2

In the second step, the largest weight of edible oil was added to a stainless steel vessel. While mixing, the remaining components were added in order of the smallest weight to the largest weight. Mixing continues until all the mixture was uniform.

Various processes for mixing and heating can be used in this step of the process. Particularly preferred processes suitable for use herein include, but are not limited to, various shapes of tanks, heating systems like water, steam and glycol systems and different agitators such as blades, propellers, impellers and homogenizers. Other commercial equipment having similar capabilities are known to those skilled in the art. It should be appreciated that such equipment may be suitable to provide all or part of the preparation of the present invention.

### Steps 3 to 6

Vessels containing the gelatin mass and capsule fill were staged by the encapsulation machine. The molten gelatin was pumped to the gelatin spreader boxes on the encapsulation machine and two uniform ribbons of gelatin are formed. The ribbons were stretched over a series of rollers and then over a set of dies that determine the size and shape of the capsules. The fill was pumped from a tank to a positive displacement pump, which accurately injects the fill between the two gelatin ribbons prior to them sealing together through the application of heat and pressure. The resultant capsule was incredibly flexible and elastic due to the water added to the gel mass during step 1.

Semi-transparent capsules fall from the machine and pass along a conveyor into tumble dryers. Approximately 25% to 50% of the moisture added in step 1 was removed during the tumble drying. The semi-transparent capsules were transferred onto trays, stacked and transferred into a drying room. Dry air was forced through the trays to remove the remaining free moisture from the semi-transparent capsules and complete the drying process.

Once dried, the semi-transparent capsules were removed from the drying room so as to undergo visual inspection. The capsules were sized, polished and packed into a polyethylene bag and placed in a labeled, shipping carton.

In practicing the process disclosed herein, the ribbon thickness in the present invention has a critical effect on releasing taste, flavor, aroma, mouth-feel and after-effect of the capsule fill. The ribbon thickness should generally between 0.030 and 0.090 cm, preferably 0.050 to 0.080 cm and more preferably 0.060 to 0.070 cm. Additionally, the gelatin to plasticizer ratio used in the practice of this invention can be in a 1:1 ratio. However, routine experimentation may be necessary in order to establish the optimum ratio for a given set of gelatin to plasticizer. Furthermore, it should be noted that as the drying proceeds, the water evaporates from the gelatin shell, without increasing the capsule shell hardness. In fact, capsule hardness does not significantly change from 2.5 Newtons throughout the process, as measured by a Bareiss hardness tester. Finally, the semi-transparent appearance of the capsule shell as a result of the oil soluble flavor, allows the consumer to have a capsule which is visually elegant and aesthetically appealing.

Tables 1 and 2 summarize compositions of flavored, chewable soft gelatin capsule produced in accordance with the methods described in Example 1.

TABLE 1

| | |
|---|---|
| | Quantity |
| Description | (Kg) |

Gelatin mass

| | |
|---|---|
| Gelatin | 32.7 |
| Glycerin | 32.7 |
| Water | 27.3 |
| Xylitol | 4.6 |
| Powdered cellulose | 2.7 |
| Total | 100.0 |

Blended Gel mass

| | |
|---|---|
| Gelatin mass | 99.6 |
| Cinnamon flavor | 0.4 |
| Total | 100.0 |

Capsule Fill

| | |
|---|---|
| Flax oil | 49.8 |
| Cinnamon flavor | 0.2 |
| Total | 50.0 |

TABLE 2

| | | |
|---|---|---|
| | Formulation 1 | Formulation 2 |
| | Quantity | Quantity |
| Description | (kg) | (kg) |

Gelatin mass

| | | |
|---|---|---|
| Gelatin | 32.7 | 32.7 |
| Glycerin | 32.7 | 32.7 |
| Water | 27.3 | 27.3 |
| Xylitol | 4.6 | 4.6 |
| Powdered cellulose | 2.7 | 2.7 |
| Total | 100.0 | 100.0 |

Blended Gel mass

| | | |
|---|---|---|
| Gelatin mass | 94.7 | 96.4 |
| Lemon flavor | — | 1.4 |
| Strawberry flavor | — | 1.2 |
| Orange flavor | — | 1.0 |
| Apple flavor | 3.5 | — |
| Vanilla flavor | 1.4 | — |
| Cinnamon flavor | 0.4 | — |
| Total | 100.0 | 100.0 |

Capsule fill

| | | |
|---|---|---|
| Borage oil | — | 48.2 |
| Flax Oil | 47.4 | — |
| Lemon flavor | — | 0.7 |
| Strawberry flavor | — | 0.6 |
| Orange flavor | — | 0.5 |
| Apple flavor | 1.7 | — |
| Vanilla flavor | 0.7 | — |
| Cinnamon flavor | 0.2 | — |
| Total | 50.0 | 50.0 |

### Example 2

### Alternative Method for Manufacture of Gelatin Capsule

Step (1): In a gelatin reactor, water, plasticizer, disintegrant and sweetening agent were added. Under vacuum and agitation, the mixture was heated to approximately 65° C. Gelatin was added and the agitation and vacuum continued for an additional 45±15 minutes. The gelatin mass was subsequently dropped into receivers. With a turbine mixer, the oil soluble flavor and colorant (optional) were added and blended until uniform.

Step (2): Approximately 80% remaining edible oil, sweetening agent, oil soluble flavor and oil soluble colorant (optional) were added to a stainless steel tank. To a second stainless steel tank, approximately 20% of the edible oil and thickening agent were added and heated to approximately 60° C. until all the material was melted. The mixer was then started and the entire quantity of the mixture from the second tank was slowly added to the first tank. When the addition was complete, the contents were mixed for 15±5 minutes.

Step (3): The gelatin mass and the capsule fill were set up on a rotary die encapsulation machine. The equipment settings were: 38° C. to 45° C. (spreader boxes), 8° C. to 25° C. (casting drums); 0.055 to 0.072 cm (ribbon thickness); mineral oil (ribbon lubricant); 28° C. to 38° C. (wedge temperature); 442×10 oval cavities (die configuration); and 3.5 to 8 rpm (encapsulation speed).

Step (4): The wet capsules were tumble dried for approximately 2±0.5 hours and transferred to trays and dried at 25° C. to 32° C. in a drying tunnel for not less than 16 hours.

Step (5): Each stack of dry capsules were tested for hardness (no more than 2.5 Newtons), inspected for acceptable quality limits (AQL), sized and polished.

Step (6): The finished capsules were packed into a polyethylene bag and placed in a labeled, shipping carton. The finished product was sampled, tested and released by quality.

Table 3 summarizes compositions of a flavored, chewable soft gelatin capsule that was prepared using the alternate method described in Example 2.

TABLE 3

| | | |
|---|---|---|
| | Formulation 1 | Formulation 2 |
| | Quantity | Quantity |
| Description | (kg) | (kg) |

Gelatin mass

| | | |
|---|---|---|
| Gelatin | 32.7 | 32.7 |
| Glycerin | 32.7 | 32.7 |
| Water | 27.3 | 27.3 |
| Xylitol | 4.6 | 4.6 |
| Powdered cellulose | 2.7 | 2.7 |
| Total | 100.0 | 100.0 |

Blend Gel mass

| | | |
|---|---|---|
| Gelatin mass | 96.5 | 96.5 |
| Orange flavor | 3.5 | 3.5 |
| Water | 100.0 | 100.0 |

Capsule fill

| | | |
|---|---|---|
| Borage oil | 22.3 | — |
| Un-winterized tuna oil | — | 19.1 |
| Lutein | — | 3.7 |
| Beeswax | 2.2 | 1.8 |
| Sucrolose | 0.005 | — |
| Orange flavor | 0.5 | 0.4 |
| Total | 25.005 | 25.0 |

### Equivalents

Those skilled in the art will recognize, or be able to ascertain using no more than routine experimentation, many equivalents to the specific embodiments of the invention described herein. Such equivalents are intended to be encompassed by the following claims.

1-44. (canceled)

45. A gelatin capsule shell for use in a pharmaceutical or nutraceutical composition, comprising an oil soluble flavoring agent.

46. The shell of claim 45, wherein the shell has a hardness of less than 4, 3, 2 or 1 Newtons (as measured on a Bareiss hardness tester).

47. The shell of claim 45, wherein the shell has a hardness of between 0.5 and 4 Newtons, between 0.5 and 3 Newtons or between 0.75 and 2 Newtons.

48. The shell of claim 45, wherein the shell comprises gelatin and a plasticizer.

49. The shell of claim 48, wherein the plasticizer is selected from the group consisting of glycerin, propylene glycol, polyethylene glycol, sorbitol, maltitol and combinations thereof.

50. The shell of claim 48, wherein the plasticizer is glycerin.

51. The shell of claim 48, wherein the gelatin and plasticizer are present at a ratio of between 1.5:1 to 0.5:1, of between 1.25:1 to 0.75:1, or of between 1.10:1 to 0.9:1.

52. The shell of claim 48, wherein the shell comprises between 25% and 60% by weight, between 35% and 55% by weight, or between 40% and 50% by weight of plasticizer.

53. The shell of claim 45, wherein the shell is semi-transparent.

54. The shell of claim 45, wherein the shell is semi-opaque.

55. The shell of claim 45, wherein the shell comprises between 0.01% and 15% by weight of oil soluble flavoring agent, between 0.1% and 12% by weight of oil soluble flavoring agent, between 0.4% and 10% by weight of oil soluble flavoring agent or between 2% and 8% by weight of oil soluble flavoring agent.

56. The shell of claim 45, wherein the oil soluble flavoring agent comprises a flavor selected from the group consisting of grape, apple, cinnamon, berry, strawberry, orange, cherry, lemon, lime, raspberry, peach, grapefruit, mango, guava, mint, chocolate, coca, vanilla, lemon, nut, almond, coconut, blueberry, blackberry, banana, pineapple and combinations thereof.

57. The shell of claim 45, wherein the shell comprises between 25% and 45% by weight, between 35% and 45% by weight, or between 40% and 45% by weight of gelatin.

58. The shell of claim 45, wherein the gelatin has a single bloom of between 100 and 275, between 150 and 200, or between 160 and 180.

59. The shell of claim 45, further comprising at least one of plasticizer, water, disintegrant, sweetening agent and colorant.

60. The shell of claim 45, wherein the shell comprises a disintegrant selected from the group consisting of cellulose, cellulose derivative, starch, starch derivative, gum, gum derivative, clay, polysaccharide, polyvinylpyrrolidone derivative, silica and combinations thereof.

61. The shell of claim 45, wherein the shell comprises between 1% and 10% by weight, between 2.5% and 7.5% by weight, or between 4% and 6% by weight of disintegrant.

62. The shell of claim 45, wherein the shell comprises between 1% and 15% by weight, between 3% and 10% by weight or between 5% and 7% by weight of water (as calculated from loss-on-drying).

63. The shell of claim 45, wherein the shell comprises a sweetening agent selected from the group consisting of monosaccharide sugars, disaccharide sugars, polysaccharide sugars, sugar alcohols, mannitol, xylitol, maltitol, sorbitol, synthetic sugar substitutes, aspartame, saccharine, sodium sacchrinate, acesulpham K, neohesperidin hydrochloride, sodium cyclamate, naturally occurring sugar substitutes, sucralose, and combinations thereof.

64. The shell of claim 45, wherein the shell comprises between 0.1% and 25% by weight, between 5% and 20% by weight, or between 10% and 15% by weight of sweetening agent (as calculated from the dry weight basis).

65. The shell of claim 45, wherein the shell is a chewable shell.

66. The shell of claim 45, wherein the shell is colored.

67. The shell of claim 45, wherein the shell comprises a colorant selected from the group consisting of natural colorants, synthetic colorants and combinations thereof.

68. A gelatin capsule comprising the shell of claim 45.

69. A method of producing a gelatin capsule shell mass comprising
(a) heating a mixture of gelatin and a plasticizer under vacuum; and
(b) adding an oil soluble flavoring agent to the heated mixture.

70. The method of claim 69, wherein the plasticizer is selected from the group consisting of glycerin, propylene glycol, polyethylene glycol, sorbitol, maltitol and combinations thereof.

71. The method of claim 69, wherein the plasticizer is glycerin.

72. The method of claim 69, wherein the mixture comprises gelatin and plasticizer at a ratio of between 1.5:1 to 0.5:1, between 1.25:1 to 0.75:1 or between 1.1:1 to 0.9:1.

73. The method of claim 69, wherein the gelatin capsule shell mass comprises between 0.01% and 15%, between 0.1% and 12% by weight of oil soluble flavoring agent, between 0.4% and 10% or between 2% and 8% by weight of oil soluble flavoring agent.

74. The method of claim 69, further comprising adding a disintegrant to the gelatin and glycerin mixture, wherein the disintegrant is selected from the group consisting of cellulose, cellulose derivative, starch, starch derivative, gum, gum derivative, clay, polysaccharide, polyvinylpyrrolidone derivative, silica and combinations thereof.

75. The method of claim 74, wherein the gelatin capsule shell mass comprises between 1% and 10% by weight, between 2.5% and 7.5% by weight, or between 4% and 6% by weight of disintegrant.

76. The method of claim 74, wherein the disintegrant comprises cellulose.
